(19) United States
(12) Patent Application Publication
(10) Pub. No.: US 2025/0049475 A1
(43) Pub. Date: Feb. 13, 2025

(54) POSTERIOR LATERAL OFFSET BASED SPINAL CORRECTION SYSTEM

(71) Applicant: The Children's Hospital of Philadelphia, Philadelphia, PA (US)

(72) Inventors: Patrick Cahill, Merion Station, PA (US); Benjamin Sinder, Philadelphia, PA (US); Jason Anari, Villanova, PA (US); Thomas P. Schaer, Landenberg, PA (US); Brian Snyder, Westwood, MA (US)

(73) Assignee: The Children's Hospital of Philadelphia, Philadelphia, PA (US)

(21) Appl. No.: 18/294,733

(22) PCT Filed: Aug. 2, 2022
(86) PCT No.: PCT/US2022/039172

§ 371 Date: Feb. 2, 2024

Related U.S. Application Data
(60) Provisional application No. 63/228,229, filed on Aug. 2, 2021.

(51) Int. Cl. A61B 17/70; A61B 17/56

(52) U.S. Cl. CPC A61B 17/7014; A61B 17/7022; A61B 2017/564

(57) ABSTRACT

A method for treating scoliosis in a patient is disclosed. The method comprises identifying a spinal column having a lateral convex curve; providing a first anchor, a second anchor, and a tether; securing the first anchor to a posterior region of a first vertebra; securing the second anchor to a posterior region of a second vertebra; securing a first tether end at a first tether connection location; and securing a second tether end at a second tether connection location. A scoliosis treatment system comprises anchors, each having a tether connection location positioned at a first location offset in a first lateral direction from a midsagittal plane of the first or second vertebra, and the anchors do not include any tether connection locations offset in in a second lateral direction, opposite the first lateral direction, from the midsagittal plane of the first vertebra or second vertebra.

### CROSS REFERENCE TO RELATED APPLICATIONS

This application claims priority from U.S. Provisional Application Ser. No. 63/228,229, titled POSTERIOR LATERAL OFFSET BASED SPINAL CORRECTION SYSTEM, filed Aug. 2, 2021, which is incorporated herein by reference in its entirety.

### FIELD OF THE INVENTION

The present invention relates generally to orthopedic devices and treatments, and more particularly, to scoliosis treatment systems and methods for treating scoliosis in a patient.

### BACKGROUND OF THE INVENTION

Scoliosis is a deformity related to an abnormal lateral curvature of the spine, such that the spinal column is bent or twisted and/or the body is tilted left or right. Scoliosis is a common diagnosis in childhood or early adolescence, i.e. during growth spurts or puberty.

Orthopedic devices and methods of treatment for scoliosis are configured to mitigate or correct deformities of the spinal column by selective application of forces. In growing patients, growth friendly non-fusion scoliosis treatment systems and methods can accommodate the growth period of the patient.

Conventional growth friendly devices and methods of treatment for scoliosis include non-fusion based scoliosis treatment strategies that focus on anterior vertebral body tethering (AVBT). In AVBT systems, screws or anchors are placed transversely in at least two vertebrae then connected with a tether rope on the long side of the spinal curve. Example AVBT devices may include The Tether™—Vertebral Body Tethering System, as designed and manufactured by Zimmer Biomet Spine, Inc. of Westminster, Colorado. The location of the tether forces in such AVBT devices are anterior to the midline of the spine due to the anterior placement of the screws or anchors. Accordingly, use of AVBT in scoliosis patients may cause or exacerbate an existing condition of kyphotic bend or kyphosis (excessive outward curvature of the spine, causing a hunchback or slouching posture), such that use of AVBT may incur a negative or neutral effect relative to the deformities of the patient's spinal column.

Further, correction of spinal curvature (such as with use of tethering systems like AVBT) requires regulating tether tension. In particular, the in vivo tether tension over time affects a patient's adaptive response to the spinal correction strategy.

The inventors have discovered that variability in tether tension in vivo may be due to dynamic loading from patient motion, patient growth, and viscoelasticity of the patient's spine. For example, the inventors performed a wireless electronic measurement of tether tension in vivo. In this study, a calibrated strain gauge, such as gauge 1500, was attached to a custom lateral offset connector, such as anchors 1100, 1200, and a posterior based tether construct with the flexible metal tether cable, such as tether 1300, spanning T11-L4 was implanted into a rapidly growing Yorkshire pig. The strain gauge 1500 was connected to an implanted electronic board (having a wireless transmitter 1600) with battery power inside a custom 3D printed case. Tether tension data was remotely recorded during surgery (8 Hz) and during rest and ambulation (32 Hz), three times per week for two weeks. Synchronized video further aligned animal activity state with recorded tether tensions values.

The results indicated that after imparting tether tension during surgery (100N) to induce a mild deformity, a rapid (over ˜1-4 hours) decline in tether tension likely related to viscoelasticity of the spine was observed. In vivo data over time revealed that ambulation tended to produce tether tensions with a range of approximately 50N from peak to trough. Peak tether tension was observed during rapid movements. Conversely, tether tension while standing and lying down values were near zero. Synchronized video and high frequency (32 Hz) tether tension data show correlation of movement with tether forces. Accordingly, tether tension was observed to decline post-surgery and is highly dynamic with movement of the patient.

Thus, improved systems and devices are desired for simultaneously maintaining or improving management of kyphosis and scoliosis, in view of variability introduced by at least patient growth and movement.

The foregoing description of the background of the invention is not an admission that all knowledge described in the background is prior art.

### SUMMARY OF THE INVENTION

In accordance with one aspect, a method for treating scoliosis in a patient is disclosed. The method comprises identifying a spinal column having a lateral convex curve extending in a first lateral direction from a midsagittal plane of the patient; providing a first anchor, a second anchor, and a tether; securing the first anchor to a posterior region of a first vertebra of the spinal column; securing the second anchor to a posterior region of a second vertebra of the spinal column; securing a first tether end of the first anchor at a first tether connection location located in the first lateral direction from a midsagittal plane of the first vertebra; and securing a second tether end to the second anchor at a second tether connection location located in the first lateral direction from a midsagittal plane of the second vertebra.

In accordance with another aspect, a method for treating scoliosis in a patient is disclosed. The method comprises installing a tether between a first vertebra of spinal column of the patient and a second vertebra of a spinal column of the patient, wherein the tether is offset on a first lateral side of a midsagittal plane of the first vertebra and offset on a first lateral side of a midsagittal plane of the second vertebra, with the first lateral side of a midsagittal plane of the first vertebra and the first lateral side of a midsagittal plane of the second vertebra are offset in a common lateral direction relative to a midsagittal plane of the patient; and generating a variable resting tensile force in the tether by a combination of increasing a distance between the first vertebra and the second vertebra, and changing a length of a variable force member installed between the tether and the first vertebra.

In accordance with yet another aspect, a scoliosis treatment system is disclosed. The system comprises a first anchor configured to install on a posterior region of a first vertebra. The first anchor comprises one or more first anchor locations at which the first anchor is configured to secure to the first vertebra, and a first offset connector portion fixedly connected to the one or more first anchor locations and extending from the one or more first anchor locations to a first tether connection location. The first anchor is configured such that, when the one or more first anchor locations are secured to the first vertebra: the first tether connection location is positioned at a first location offset in a first lateral direction from a midsagittal plane of the first vertebra, and the first anchor does not include any tether connection locations offset in in a second lateral direction, opposite the first lateral direction, from the midsagittal plane of the first vertebra. The system also comprises a second anchor configured to install on a posterior region of a second vertebra. The second anchor comprises one or more second anchor locations at which the second anchor is configured to secure to the second vertebra, and a second offset connector portion fixedly connected to the one or more second anchor locations and extending from the one or more second anchor locations to a second tether connection location. The second anchor is configured such that, when the one or more second anchor locations are secured to the second vertebra: the second tether connection location is positioned at a second location offset in a second lateral direction from a midsagittal plane of the second vertebra, and the second anchor does not include any tether connection locations offset in in a second lateral direction, opposite the second lateral direction, from the midsagittal plane of the second vertebra. The system further comprises a tether configured to attach to and extend between the first tether connection location and the second tether connection location.

In accordance with an aspect, a scoliosis treatment system is disclosed. The system comprises a first anchor configured to install on a first vertebra. The first anchor comprises one or more first anchor locations at which the first anchor is configured to secure to the first vertebra, and a first offset connector portion fixedly connected to the one or more first anchor locations and extending from the one or more first anchor locations to a first tether connection location. The first anchor is configured such that, when the one or more first anchor locations are secured to the first vertebra, the first tether connection location is positioned at a first location offset in a first lateral direction from a midsagittal plane of the first vertebra. The system also comprises a second anchor configured to install on a second vertebra. The second anchor comprises one or more second anchor locations at which the second anchor is configured to secure to the second vertebra, and a second offset connector portion fixedly connected to the one or more second anchor locations and extending from the one or more second anchor locations to a second tether connection location. The second anchor is configured such that, when the one or more second anchor locations are secured to the second vertebra, the second tether connection location is positioned at a second location offset in a second lateral direction from a midsagittal plane of the second vertebra. Further, the system comprises a tether extending from a first tether end configured to be slidably secured within the first tether connection location, to a second tether end configured to be secured at the second tether connection location.

In accordance with another aspect, a method for treating scoliosis in a patient is disclosed. The method comprises identifying a spinal column having a lateral convex curve extending in a first lateral direction from a midsagittal plane of the patient; providing a first anchor, a second anchor and a tether; securing the first anchor to a first vertebra of the spinal column; securing the second anchor to a second vertebra of the spinal column; slidably securing a first tether end to the first anchor at a first tether connection location located in the first lateral direction from a midsagittal plane of the first vertebra; and securing a second tether end to the second anchor at a second tether connection location located in the first lateral direction from a midsagittal plane of the second vertebra.

In accordance with yet another aspect, a scoliosis treatment system is disclosed. The system comprises a first anchor configured to install on a first vertebra. The first anchor comprises one or more first anchor locations at which the first anchor is configured to secure to the first vertebra, and a first offset connector portion fixedly connected to the one or more first anchor locations and extending from the one or more first anchor locations to a first tether connection location. The first anchor is configured such that, when the one or more first anchor locations are secured to the first vertebra, the first tether connection location is positioned at a first location offset in a first lateral direction from a midsagittal plane of the first vertebra. The system also includes a second anchor configured to install on a second vertebra. The second anchor comprises one or more second anchor locations at which the second anchor is configured to secure to the second vertebra, and a second offset connector portion fixedly connected to the one or more second anchor locations and extending from the one or more second anchor locations to a second tether connection location. The second anchor is configured such that, when the one or more second anchor locations are secured to the second vertebra, the second tether connection location is positioned at a second location offset in a second lateral direction from a midsagittal plane of the second vertebra. The system further comprises a tether assembly extending from a first tether end configured to be secured at the first tether connection location, to a second tether end configured to be secured at the second tether connection location. The tether assembly comprises a tether, and a spring operationally secured between the tether and the first tether connection location to compress or expand as a function of tension in the tether.

In accordance with an aspect, a method for treating scoliosis in a patient is disclosed. The method comprises identifying a spinal column having a lateral convex curve extending in a first lateral direction from a midsagittal plane of the patient; providing a first anchor, a second anchor and a tether assembly comprising a tether and a spring; securing the first anchor to a first vertebra of the spinal column; securing the second anchor to a second vertebra of the spinal column; securing the tether assembly to the first anchor at a first tether connection location located in the first lateral direction from a midsagittal plane of the first vertebra; and securing the tether assembly to the second anchor at a second tether connection location located in the first lateral direction from a midsagittal plane of the second vertebra.

In accordance with an aspect, a scoliosis treatment system is disclosed. The system comprises a first anchor configured to install on a first vertebra. The first anchor includes one or more first anchor locations at which the first anchor is configured to secure to the first vertebra, and a first offset connector portion fixedly connected to the one or more first anchor locations and extending from the one or more first anchor locations to a first tether connection location. The first anchor is configured such that, when the one or more first anchor locations are secured to the first vertebra, the first tether connection location is positioned at a first location offset in a first lateral direction from a midsagittal plane of the first vertebra. The system also includes a second anchor configured to install on a second vertebra. The second anchor comprises one or more second anchor locations at which the second anchor is configured to secure to the second vertebra, and a second offset connector portion fixedly connected to the one or more second anchor locations and extending from the one or more second anchor locations to a second tether connection location. The second anchor is configured such that, when the one or more second anchor locations are secured to the second vertebra, the second tether connection location is positioned at a second location offset in a second lateral direction from a midsagittal plane of the second vertebra. The system further comprises a tether extending from a first tether end configured to be secured at the first tether connection location, to a second tether end configured to be secured at the second tether connection location. The system also includes a sensor system configured to generate a signal representing a tension in the tether.

In accordance with an aspect, a method for treating scoliosis in a patient is disclosed. The method comprises identifying a spinal column having a lateral convex curve extending in a first lateral direction from a midsagittal plane of the patient; providing a first anchor, a second anchor, a tether, and a sensor system configured to generate a signal representing a tension in the tether; securing the first anchor to a first vertebra of the spinal column; securing the second anchor to a second vertebra of the spinal column; securing the tether to the first anchor at a first tether connection location located in the first lateral direction from a midsagittal plane of the first vertebra; securing the tether to the second anchor at a second tether connection location located in the first lateral direction from a midsagittal plane of the second vertebra; and monitoring the sensor system during a growth period of the patient.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. According to common practice, the various features of the drawings are not drawn to scale unless otherwise indicated. On the contrary, the dimensions of the various features may be expanded or reduced for clarity.

FIGS. 1A-1B depict images depicting an exemplary scoliosis treatment system, in accordance with an embodiment of the invention.

FIG. 2 depict images depicting another exemplary scoliosis treatment system, in accordance with an embodiment of the invention.

FIG. 3 is a schematic diagram depicting still another exemplary scoliosis treatment system, in accordance with an embodiment of the invention.

FIG. 4 depicts an exemplary method of treating scoliosis in a patient, in accordance with an embodiment of the invention.

FIG. 5 depicts another exemplary method of treating scoliosis in a patient, in accordance with an embodiment of the invention.

FIG. 6 depicts yet another exemplary method of treating scoliosis in a patient, in accordance with an embodiment of the invention.

FIG. 7 depicts still another exemplary method of treating scoliosis in a patient, in accordance with an embodiment of the invention.

FIG. 8 depicts another exemplary method of treating scoliosis in a patient, in accordance with an embodiment of the invention.

FIG. 9 are images depicting results of an animal model study for studying spinal curvature correction.

### DETAILED DESCRIPTION OF THE INVENTION

Aspects of the invention are described herein with reference to medical care and management of spinal deformities, including a spinal column having a lateral convex curve as seen in patients with scoliosis and/or kyphosis. It will be understood by one of ordinary skill in the art that the exemplary devices, methods of treatment, and treatment systems described herein are not limited to patients who have specific scoliosis and/or kyphosis conditions, or to specific care and management protocols. Other types of patients or medical treatment plans suitable for use with the disclosed devices, methods of treatment, and treatment systems will be known to one of ordinary skill in the art from the description herein.

A “patient” as described herein is an individual having characteristics at least within a known or expected range of values, which may be actual values as measured, or expected ranges based upon characteristic(s) of a scoliosis and/or kyphosis condition, as well as birth gender, age, height, weight, growth rate, bone growth and development, and other physiological factors. It should be understood that devices, methods of treatment, and treatment systems intended for a patient may be sized or designed to accommodate a specific individual patient or a spectrum of patients having characteristics of a scoliosis and/or kyphosis condition, and physiological measurements within the expected ranges of values. As used throughout the specification, the term “characteristic(s) of a scoliosis and/or kyphosis” is intended to encompass various characteristics of spinal deformities, such as spinal deformities that may be mild or progressive (worsen over time), may be associated with other health conditions, such as inflammation, etc. (as a result of said spinal deformities or otherwise), may cause a degree of pain and/or disability, and/or may require some level of corrective treatment.

While the exemplary embodiments of the invention are described herein with respect to methods and systems for treating scoliosis using a posterior spinal approach, it will be understood that the invention is not necessarily limited to the posterior approach. For example, certain aspects may be used in methods and systems that use an anterior spinal approach, such as existing devices for treatment of scoliosis and/or kyphosis (such as AVBT systems). Other suitable applications will be readily understood by one of ordinary skill in the art from the description herein.

Generally, an exemplary scoliosis treatment system 1000 comprises a first anchor 1100, a second anchor 1200, and a tether or tether assembly 1300. First anchor 1100 is configured to be secured to a first vertebra of a spinal column of the patient and second anchor 1200 is configured to be secured to a second vertebra of the spinal column of the patient. First anchor 1100 and second anchor 1200 are shown in this embodiment being installed on respective posterior regions of the first vertebra and the second vertebra of the spinal column, but this is not strictly necessary in all embodiments. Tether or tether assembly 1300 is configured to attach to and extend between a portion of first anchor 1100 and a portion of second anchor 1200. Additional details of system 1000 will now be described below.

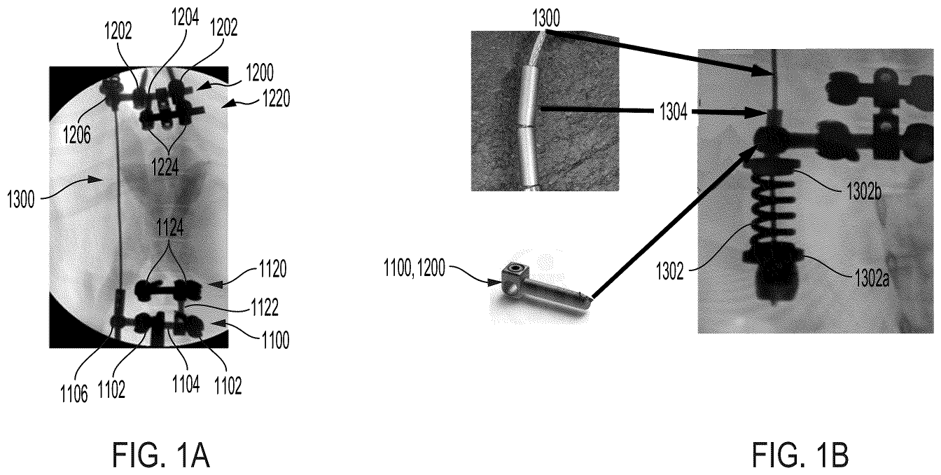

Referring now to FIGS. 1A-1B, first anchor 1100 of system 1000 is a generally beam-like structure having one or more first anchor locations 1102 at which first anchor 1100 is configured to secure to the first vertebra of the spinal column, and a first offset connector portion 1104 fixedly connected to one or more first anchor locations 1102. First anchor 1100 may include, for example, lateral connectors or lateral offset connectors, such as those of the EXPEDIUM Spine System, as designed and manufactured by DePuy Synthes Spine, Inc. of Raynham, Massachusetts; those of the CD HORIZON SOLERA 5.5/6.0 SPINAL SYSTEM, as designed and manufactured by Medtronic SOFAMOR DANEK of Memphis, Tennessee; and those of the MOUNTAINEER® Occipito-Cervico-Thoracic Spinal System, as designed and manufactured by DePuy spine, Inc. of Raynham, Massachusetts, or DePuy Spine SÀRL of Le Locle, Switzerland, or Medos International SÀRL of Le Locle, Switzerland. As used herein and throughout the specification, the term “fixedly connected” or “fixedly” is intended to mean the absence of appreciable relative movement (such as between first offset connector portion and one or more first anchor locations) when subject components are connected or attached. In this case, the first anchor 1100 is configured to secure to a posterior region of the first vertebra. In other cases, such as where features of the system 1000 are used in an exiting anterior spinal correction system, the first anchor location 1102 may comprise a threaded screw shaft and the first offset connector portion 1104 may comprise a head attached to the threaded screw shaft. In this configuration, the head may comprise a hole or a slot therethrough to slidably receive a first tether end 1306 of tether 1300 (discussed below).

First offset connector portion 1104 extends from one or more first anchor locations 1102 to a first tether connection location 1106. In this way, first anchor 1100 is configured such that, when the one or more first anchor locations 1102 are secured to the first vertebra, the first tether connection location 1106 is positioned at a first location offset in a first lateral direction from a midsagittal plane of the first vertebra. Additionally or optionally, first anchor 1100 does not include any tether connection locations 1106 offset in a second lateral direction, opposite the first lateral direction, from the midsagittal plane of the first vertebra. This arrangement allows the first anchor 1100 to be relatively compact and less intrusive in the patient.

The one or more first anchor locations 1102 may comprise at least two first anchor locations 1102 configured to secure to the first vertebra at two locations. In an embodiment, the two location may include opposite sides of the midsagittal plane of the first vertebra. In this case, the first tether connection location 1106 may be configured to be offset in the first lateral direction from the midsagittal plane of the first vertebra relative to all of the one or more first anchor locations 1102 when the one or more first anchor locations 1102 are secured to the first vertebra, but this is not strictly required. The first anchor locations 1102 may comprise respective first pedicle screw openings, such as openings configured to receive pedicle screws for securing first anchor 1100 to the first vertebra, or other structures suitable for securing of facilitating securement to the vertebra.

Second anchor 1200 may be substantially similar to first anchor 1100. For example, the shown second anchor 1200 comprises one or more second anchor locations 1202 at which the second anchor 1200 is configured to secure to the second vertebra, and a second offset connector portion 1204 fixedly connected to the one or more second anchor locations 1202. The second offset portion 1204 extends from the one or more second anchor locations 1202 to a second tether connection location 1206, such that the second tether connection location 1206 is positioned at a second location offset in a second lateral direction from a midsagittal plane of the second vertebra when second anchor 1200 is installed. As with first anchor 1100, second anchor 1200 may optionally not include any tether connection locations 1206 offset in in a second lateral direction, opposite the second lateral direction, from the midsagittal plane of the second vertebra, so as to reduce the size and to reduce potentially increased traumatic effect of the device on the patient.

As before, the one or more second anchor locations 1202 may include at least two second anchor locations 1202 configured to secure to the second vertebra on two locations. The two location may include opposite sides of the midsagittal plane of the second vertebra. The second tether connection location 1206 may be offset in the first lateral direction from the midsagittal plane of the second vertebra relative to all of the one or more second anchor locations 1202. The second anchor locations 1202 may comprise respective second pedicle screw openings, such as openings configured to receive pedicle screws for securing second anchor 1200 to the second vertebra, or any other suitable connection means for securing to the spine.

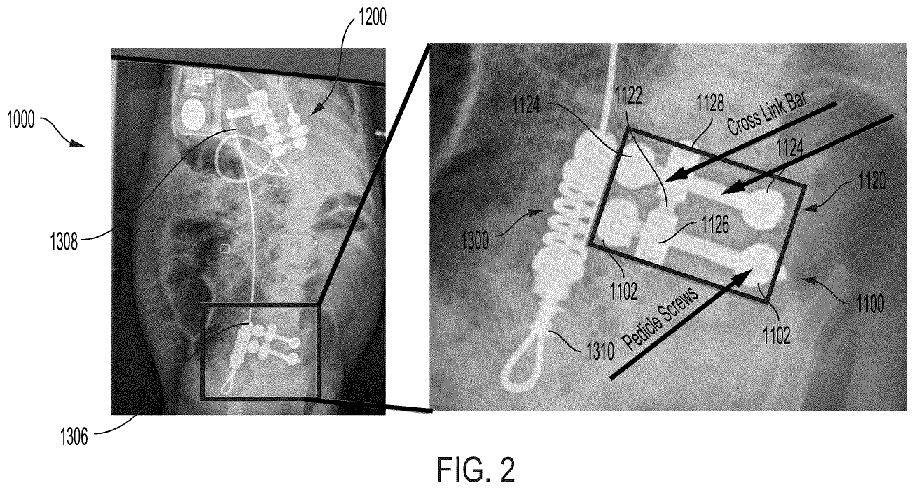

As shown in FIGS. 1A-1B and 2, system 1000 comprises tether 1300. Tether 1300 can be selected to have any suitable size and material, as determined by one or more factors, including but not limited to physiological measurements of the patient, the characteristics of the scoliosis, kyphosis, or spinal deformity of the patient, standards or treatment protocol established by medical professionals, and feature(s) of one or more components of system 1000. In an exemplary embodiment, tether 1300 may comprise a metal wire. In a non-limiting example, tether 1300 comprises a 1.77 mm metal (e.g. cobalt chrome) cable, such as a metal cable used in treating fracture trauma (non-spine). Tether 1300 comprising metal wire may provide improved resistance failure and/or stability in view of breakage risk after a spinal correction device is implanted in the patient, particularly if the patient is expected to experience bone growth post-implantation. Optionally, tether 1300 may instead comprise a braided rope, such as a polyester (PET) woven rope. Tether 1300 also may comprise other materials and structures, such as composite materials, metals of various implant-safe materials, and so on.

Tether 1300 is configured to attach to and extend between the first tether connection location 1106 and the second tether connection location 1206. More specifically, a first tether end 1306 is attached at the first tether connection location 1106, and a second tether end 1308 is attached at the second tether connection location 1206. The tether ends 1306, 1308 may be connected to the respective tether connection location 1106, 1206 by any suitable connection. In some cases, the tether ends 1306, 1308 may be fixedly connected to the respective tether connection location 1106, 1206, such that there is no relative movement of the tether end relative to the tether connection location. In other cases, one or both tether ends 1306, 1308 may be slidably secured at the respective tether connection location 1106, 1206. As used herein and throughout the specification, the term “slidably” or “slidable” is intended to mean an arrangement in which tether 1300 is not affixed in a manner that prevents or restricts movement along an axis of tether 1300, but does limit or prevent lateral movement in a plane perpendicular to the axis of tether 1300. This permits modulation of tension applied by tether 1300 and therefore improves spinal alignment of the patient, particularly while the patient undergoes the growth period and/or moves. One skilled in the art would understand from the description herein that this slidable connection may be achieved by various structures, including but not limited to a hole or a slot formed in a component of system 1000, such as anchor locations 1102, 1202.

As illustrated in FIG. 1B, first tether connection location 1106 comprises a first cannulated shaft or rod 1304, and tether 1300 is configured to be slidably positioned within the first cannulated shaft 1304. One skilled in the art would understand from the description herein that cannulated rod 1304 may have various measurements (e.g. diameter) to accommodate the size, material, or other characteristic of one or more components of system 1000, including but not limited to first anchor 1100, second anchor 1200, and tether 1300. In an exemplary embodiment, first tether connection location 1106 comprises first cannulated rod 1304 and is configured to slidably contain first tether end 1306. Cannulated rod 1304 (or any other slidable connection) may include friction-reducing materials to help prevent degradation of the components.

Additionally or optionally, system 1000 comprises a tether travel stop 1310 (FIG. 2) configured to prevent a distal portion of the first tether end 1306 from passing through the first cannulated shaft 1304 towards the second tether end 1308.

Referring now to FIGS. 1B and 2, tether 1300 may be provided as part of a tether assembly. In this case, the tether assembly comprises tether 1300 and spring 1302 (FIG. 2), such as a compression spring. One skilled in the art would understand from the description herein that spring 1302 may vary and may be selected based on the patient and according to a specific treatment protocol. Spring 1302 may be operationally secured between tether 1300 and the first tether connection location 1106 to compress or expand as a function of tension in tether 1300. In other cases, spring 1302 may be operationally secured between tether 1300 and the first tether connection location 1106 which is positionable at any point along a length of the first anchor 1100, or along the first lateral direction or second lateral direction from the midsagittal plane of the first vertebra.

In a non-limiting example, spring 1302 may have a first spring end 1302a configured to fixedly secure to the tether 1300 and a second spring end 1302b configured to be positioned between the first spring end 1302a and the first cannulated rod 1304. In this configuration, the first cannulated rod 1304 is between the spring 1302 and the second tether connection location 1206. In this case, spring 1302 comprises a compression spring located between the tether travel stop 1310 and the first cannulated shaft 1304. In other cases, spring 1302 may be a tension spring provided at an end of tether 1300, with spring 1302 located between the first tether connection location 1106 and the second tether connection location 1206.

Spring 1302 modulates the stiffness or tension of the tether 1300. For example, when using a relatively stiff or tense tether 1300, relatively small changes in displacement can result in large and difficult to predict changes in force applied by tether 1300 to the spine. A very stiff tether cable can also be highly sensitive to small changes in displacement, which may not be desirable, particularly for patients undergoing patient growth. As a result, integration of spring 1302 permits tether 1300 to adjust in view of any one of viscoelastic relaxation of the spine, patient growth, movement of the patient, and so on. Managing this variability can be critical in driving the subsequent biological response and correction of the scoliotic curve. For example, exertion of excessive force can result in over correction of scoliotic curve or cause breakage of components of system 1000. Conversely, insufficient force can result in under-correction of the scoliotic curve. Thus, the relationship between spring 1302 and tether 1300 improves or overcomes the shortcomings of conventional non-fusion strategies (when used on their own, for example) for correcting spinal curvature, which solely rely on the application of force at the time of surgery, and has less focus on application of force during post-surgery conditions (e.g. movement of the patient through daily life, growth period of the patient, etc.).

In still another exemplary embodiment, as best illustrated in FIG. 2, system 1000 comprises a first support anchor 1120 configured to be installed on a posterior region of a third vertebra. A first link 1122 may be configured to fixedly connect the first support anchor 1120 to first anchor 1100. In this configuration, first anchor 1100 comprises a first anchor body extending between at least two first anchor locations anchor locations 1102. In addition, first support anchor 1120 comprises a first support anchor body extending between at least two first support anchor locations 1124. Further, first link 1122 may include a respective first link collar 1126 configured to secure to the first anchor body between the at least two first anchor locations 1102 and a respective second link collar 1128 configured to secure to the first support anchor body between the at least two first support anchor locations 1124.

System 1000 may include a second support anchor 1220, which may be substantially similar to first support anchor 1120. Second support anchor 1220 is configured to be installed on a posterior region of a fourth vertebra. A second link (not shown) is configured to fixedly connect the second support anchor 1220 and second anchor 1200, in a similar manner as first link 1122 described above. In this configuration, second anchor comprises a second anchor body extending between at least two second anchor locations 1204. In addition, second support anchor 1220 comprises a second support anchor body extending between at least two second support anchor locations 1224. Further, second link (not shown) comprises a respective first link collar configured to secure to the second anchor body between the at least two second anchor locations 1202 and a respective second link collar configured to secure to the second support anchor body 1220 between the at least two second support anchor locations 1224.

In yet another exemplary embodiment, system 1000 comprises one or more additional anchors, such as additional anchors similar to first anchor 1100 and/or second anchor 1200 and/or their respective components thereof. For example, each of the one or more additional anchors comprise one or more respective additional anchor locations at which the respective additional anchor is configured to secure to a respective vertebra located between the first vertebra and the second vertebra. Each of the one or more additional anchors also includes a respective additional offset connector portion fixedly connected to the one or more respective additional anchor locations and extending from the one or more respective additional anchor locations to a respective additional anchor tether connection location. Further, each additional anchor is configured such that, when the one or more respective additional anchor locations are secured to the respective vertebra, the respective additional anchor tether connection location is positioned at a first location offset in a first lateral direction from a midsagittal plane of the respective vertebra. As with the other exemplary embodiments described above, tether 1300 is configured to be secured at respective locations to the respective additional anchor tether connection locations. Accordingly, tether 1300 is configured to be slidably secured to one or more of the respective additional anchor tether connection locations. Additionally or optionally, one or more of the additional anchor tether connection locations comprises a respective additional cannulated shaft, such as a cannulated shaft similar to cannulated shaft 1304, and tether 1300 is configured to be slidably positioned within each of the additional cannulated shafts. Additionally or optionally, a support anchor, such as one similar to support anchors 1120, 1220, is configured to install on a third vertebra located between the first vertebra and the second vertebra. Likewise, a support link, such as one similar to link 1122 is configured to connect to one of the first anchor 1100 and the second anchor 1200.

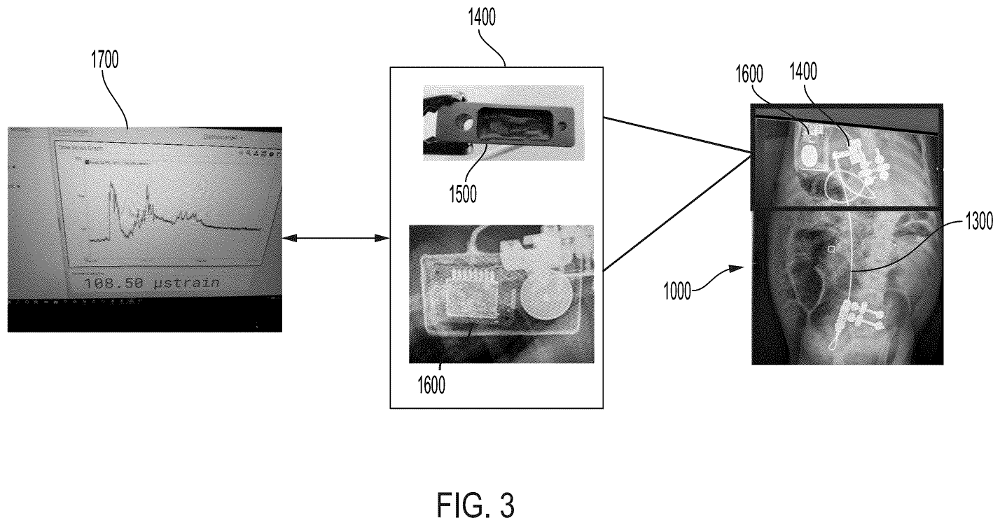

Turning now to FIG. 3, in yet an exemplary embodiment, system 1000 further comprises a sensor system 1400 configured to generate a signal representing a tension in tether 1300. To facilitate generation of the signal, sensor system 1400 comprises one or more strain gauges 1500 integrated with or attached to one or more objects, such as one or more of first anchor 1100, second anchor 1200, and tether 1300. In other cases, system 1400 comprises a Micro Electro-Mechanical System (MEMS), such as MEMS configured to assess and/or monitor real-time in vivo characteristics of the implanted components of scoliosis treatment system 1000 (e.g. anchors 1100, 1200 and/or tether 1300). The sensor system 1400 may include a processor, memory, firmware, or other computing devices or hardware as may be necessary to process input from a sensor and transmit a signal based on the input to a location outside the patient. For example, sensor system 1400 may simply include a set of wire leads that connect to an exterior or remote monitoring device. Additionally or optionally, sensor system 1400 includes a wireless transmitter 1600 configured to wirelessly send the signal to a remote monitoring device 1700. Sensor system 1400 may be powered by an internal battery, wireless power transfer, and so on.

In an exemplary embodiment, one or more strain gauges 1500 can provide a real-time, high frequency, and in vivo measurement of the spinal corrective force applied by tether 1300. To achieve this, one or more calibrated strain gauges 1500 are operatively connected the wireless transmitter 1600, which can remotely transmit data to a remote monitoring device 1700, such as a computer. This data can permit improved predictions of the biological response to the corrective treatment, subsequent growth modulation, and can also detect failure of the implanted components of system 1000, such as tether 1300.

In still another exemplary embodiment, system 1000 includes a document comprising a set of use instructions (e.g., printed instructions) or instructions for accessing the set of use instructions (e.g., instructions to scan a barcode or access a website to obtain use instructions). The set of use instructions may include instructions for: securing the first anchor 1100 to the first vertebra, such as a posterior region of the first vertebra; securing the second anchor 1200 to the second vertebra, such as a posterior region of the second vertebra; and securing the tether 1300 to the first tether connection location 1106 and the second tether connection location 1206. Other sets of instructions may include instructions for other methods as described herein or as will be apparent from the disclosure herein.

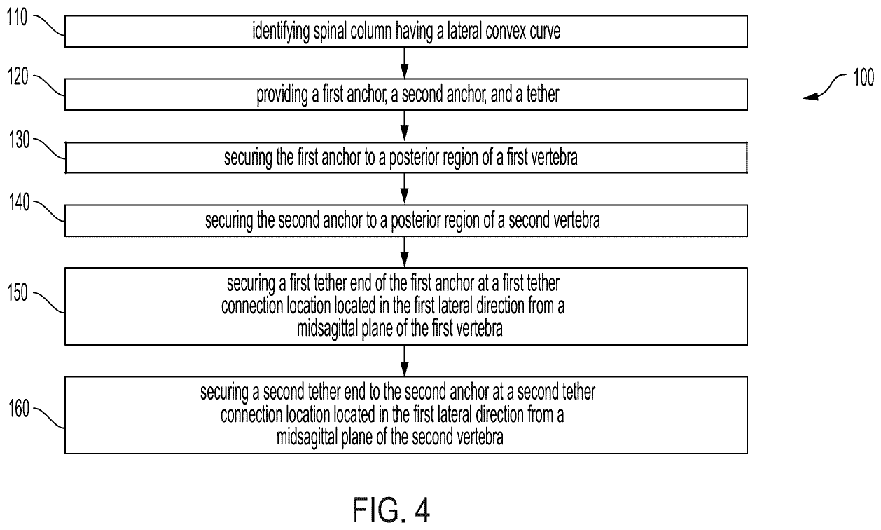

FIG. 4 illustrates an exemplary method of treating scoliosis in a patient, such as method 100, in accordance with aspects of the present invention. The method 100 includes one or more steps including identifying a spinal column having a lateral convex curve; providing a first anchor, a second anchor, and a tether; securing the first anchor to a posterior region of a first vertebra; securing the second anchor to a posterior region of a second vertebra; securing a first tether end of the first anchor at a first tether connection location located in the first lateral direction from a midsagittal plane of the first vertebra; and securing a second tether end to the second anchor at a second tether connection location located in the first lateral direction from a midsagittal plane of the second vertebra. Additional details of method 100 are set forth below with respect to the elements of scoliosis treatment system 1000. Method 100 may comprise one or more steps from any of methods 200, 300, 400, and 500 (discussed below).

In step 110, a spinal column having a lateral convex curve is identified. In particular, the lateral convex curve extends in a first lateral direction from a midsagittal plane of the patient. This lateral convex curve may be indicative of scoliosis, kyphosis, or spinal deformity.

In step 120, a first anchor, a second anchor, and a tether are provided. In an exemplary embodiment, a first anchor, such as first anchor 1100, a second anchor, such as second anchor 1200, and a tether, such as tether 1300, are provided. In particular, first anchor 1100 comprises a first anchor body and one or more first pedicle screws, the second anchor 1200 comprises a second anchor body and one or more second pedicle screws, or a combination thereof. Specifically, as discussed above, first anchor 1100 comprises a first offset connector portion 1104 extending in a lateral direction from the midsagittal plane of the first vertebra to first tether connection location 1106; and second anchor 1200 comprises a second offset connector 1204 extending in the lateral direction from the midsagittal plane of the second vertebra to second tether connection location 1206. Tether 1300 may comprise metal wire or braided rope.

In step 130, the first anchor is secured to a posterior region of a first vertebra of the spinal column. In an exemplary embodiment, first anchor 1100 is secured to the posterior region of the first vertebra of the spinal column. Specifically, securing the first anchor 1100 to the posterior region of the first vertebra comprises securing the first anchor 1100 to the pedicle of the first vertebra, securing the second anchor 1200 to the posterior region of the second vertebra comprises securing the second anchor 1200 to the pedicle of the second vertebra, or a combination thereof. In another non-limiting example, securing the first anchor 1100 to the posterior region of the first vertebra comprises securing the first anchor 1100 to the posterior region of the first vertebra in at least two locations on the first vertebra. Specifically, the at least two locations on the first vertebra comprise two locations on opposite lateral sides of a midsagittal plane of the first vertebra.

In step 140, the second anchor is secured to a posterior region of a second vertebra of the spinal column. In an exemplary embodiment, second anchor 1200 is secured to the posterior region of the second vertebra of the spinal column. Specifically, securing the first anchor 1100 to the posterior region of the first vertebra comprises securing the first anchor 1100 to the pedicle of the first vertebra, securing the second anchor 1200 to the posterior region of the second vertebra comprises securing the second anchor 1200 to the pedicle of the second vertebra, or a combination thereof. In another non-limiting example, securing the second anchor 1200 to the posterior region of the second vertebra comprises securing the second anchor 1200 to the posterior region of the second vertebra in at least two locations on the second vertebra. Specifically, the at least two locations on the second vertebra comprise two locations on opposite lateral sides of a midsagittal plane of the second vertebra.

In step 150, a first tether end of the first anchor is secured at a first tether connection location located in the first lateral direction from a midsagittal plane of the first vertebra. In an exemplary embodiment, first tether end of first anchor 1100 is secured at first tether connection location 1106 in the first lateral direction from the midsagittal plane of the first vertebra. In another exemplary embodiment, securing first tether end 1306 of tether 1300 to the first anchor 1100 comprises securing a first cannulated rod 1304 to the first anchor 1100 and slidably securing the first tether end 1306 of tether 1300 within the first cannulated rod 1304. In particular, slidably securing the first tether end 1306 within the first cannulated rod 1304 comprises: providing a spring 1302 having a first end 1302a and second end 1302b; securing a distal portion of the first tether end 1306 to the first end 1302a of the spring 1302; positioning the second end 1302b of the spring 1302 between the first end 1302a of the spring 1302 and the first cannulated rod 1304; and positioning the first cannulated rod 1304 between the second end 1302b of the spring 1302 and the second anchor 1200.

In step 160, a second tether end is secured to the second anchor at a second tether connection location located in the first lateral direction from a midsagittal plane of the second vertebra. In an exemplary embodiment, second tether end of second anchor 1200 is secured at a second tether connection location 1206 in the first lateral direction from the midsagittal plane of the second vertebra. In another exemplary embodiment, securing second tether end 1308 of tether 1300 to the second anchor 1200 comprises securing a second cannulated rod (which may be similar to first cannulated rod or shaft 1304) to the second anchor 1200 and slidably securing the second tether end 1308 of tether 1300 within the second cannulated rod.

Additionally or optionally, method 100 comprises additional steps of securing a first support anchor 1120 to a posterior region of a third vertebra of the spinal column adjacent to the first vertebra and between the first vertebra and the second vertebra; securing the first support anchor 1120 to the first anchor 1100 to inhibit rotation of the first anchor 1100 relative to the first vertebra; securing a second support anchor 1220 to a posterior region of a fourth vertebra of the spinal column adjacent to the second vertebra and between the first vertebra and the second vertebra; and securing the second support anchor 1220 to the second anchor 1200 to inhibit rotation of the second anchor 1200 relative to the second vertebra.

Still further, method 100 may additionally or optionally include steps of securing the first anchor 1100 to the posterior region of the first vertebra comprises securing the first anchor 1100 to the posterior region of the first vertebra in at least two locations on the first vertebra; securing the second anchor 1200 to the posterior region of the second vertebra comprises securing the second anchor 1200 to the posterior region of the second vertebra in at least two locations on the second vertebra; securing the first support anchor 1120 to the posterior region of the third vertebra comprises securing the first support anchor 1120 to the posterior region of the third vertebra in at least two locations on the third vertebra; and securing the second support anchor 1220 to the posterior region of the fourth vertebra comprises securing the second support anchor 1220 to the posterior region of the fourth vertebra in at least two locations on the fourth vertebra.

Additionally or optionally, method 100 includes generating a tensile force in the tether between the first anchor and the second anchor. In an exemplary embodiment, a tensile force in tether 1300 between first anchor 1100 and second anchor 1200 is generated. In particular, generating the tensile force comprises installing a spring, such as spring 1302, between tether 1300 and at least one of first anchor 1100 and second anchor 1200.

Still further, method 100 additionally or optionally includes allowing a growth period of the patient; and adjusting a tension in the tether, adjusting a length of the tether between the first anchor and the second anchor, or a combination thereof. As used herein and throughout the specification, the term “resting tension” is intended to refer to tension present when the patient's body is in a predetermined position in which relatively consistent measurements may be taken. The predetermined position may include, but is not limited to, the patient body being prone on a horizontal surface without actively applying muscle tension to the spine and/or with the patient in a sedated state. In an exemplary embodiment, tension in tether 1300 is adjusted, length of tether 1300 between first anchor 1100 and second anchor 1200 is adjusted, or a combination thereof. Still further, method 100 may additionally or optionally include periodically or continuously monitoring a resting tension in or a length of the tether 1300 during a growth period of the patient. In particular, a resting tension in or a length of tether 1300 is periodically or continuously monitored during a growth period of the patient. Monitoring the resting tension in or length of tether 1300 may comprise wirelessly communicating with a control unit (such as strain gauge 1500) operatively connected to tether 1300.

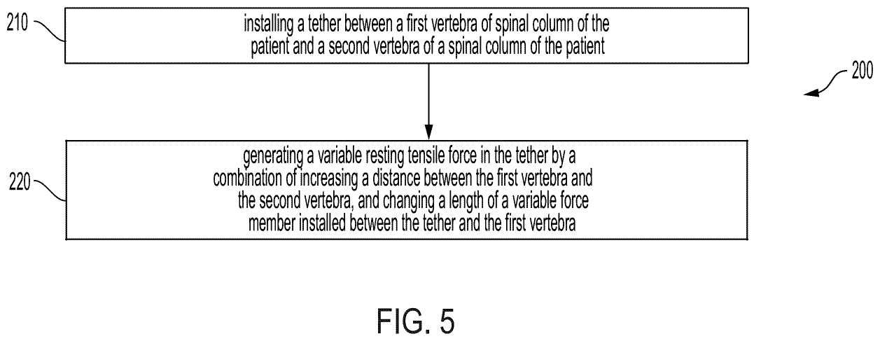

FIG. 5 illustrates another exemplary method of treating scoliosis in a patient, such as method 200, in accordance with aspects of the present invention. Method 200 is generally similar to method 100, and may include one or more steps of methods 100, 300, 400, and 500 but is different in some respects. In particular, method 200 includes one or more steps including installing a tether between a first vertebra of spinal column of the patient and a second vertebra of a spinal column of the patient; and generating a variable resting tensile force in the tether by a combination of increasing a distance between the first vertebra and the second vertebra, and changing a length of a variable force member installed between the tether and the first vertebra. Additional details of method 200 are set forth below with respect to the elements of scoliosis treatment system 1000.

In step 210, a tether between a first vertebra of spinal column of the patient and a second vertebra of a spinal column of the patient is installed. In an exemplary embodiment, tether 1300 between the first vertebra and the second vertebra is installed. Tether 1300 may be offset on a first lateral side of a midsagittal plane of the first vertebra and offset on a first lateral side of a midsagittal plane of the second vertebra, with the first lateral side of a midsagittal plane of the first vertebra. and the first lateral side of a midsagittal plane of the second vertebra are offset in a common lateral direction relative to a midsagittal plane of the patient.

In step 220, a variable resting tensile force in the tether is generated by a combination of increasing a distance between the first vertebra and the second vertebra, and changing a length of a variable force member installed between the tether and the first vertebra. In an exemplary embodiment, the variable resting tensile force in the tether 1300 is generated by a combination of increasing a distance between the first vertebra and the second vertebra, and changing a length of a variable force member installed between tether 1300 and the first vertebra. In a non-limiting example, the variable force member comprises a spring, such as spring 1302, mounted in compression between tether 1300 and the first vertebra. Additionally or optionally, method 200 comprises monitoring the variable resting tensile force during a growth period of the patient. In an exemplary embodiment, monitoring the variable resting tensile force comprises wirelessly communicating with a control unit operatively connected to tether 1300. In a non-limiting example, the control unit may comprise a strain gauge 1500 connected to an anchor 1100, 1200 securing tether 1300 to the first vertebra or the second vertebra.

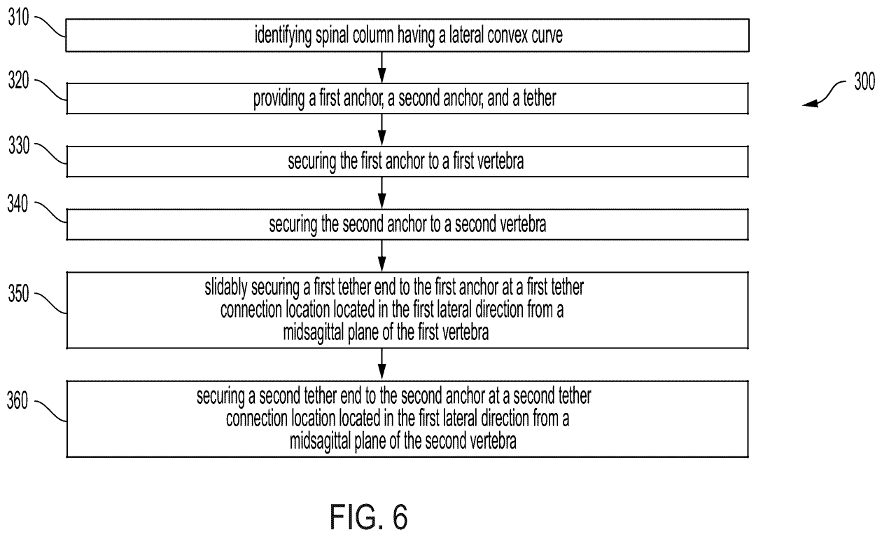

FIG. 6 illustrates another exemplary method of treating scoliosis in a patient, such as method 300, in accordance with aspects of the present invention. In particular, method 300 includes one or more steps including identifying a spinal column having a lateral convex curve; providing a first anchor, a second anchor and a tether; securing the first anchor to a first vertebra of the spinal column; securing the second anchor to a second vertebra of the spinal column; slidably securing a first tether end to the first anchor at a first tether connection location located in the first lateral direction from a midsagittal plane of the first vertebra; and securing a second tether end to the second anchor at a second tether connection location located in the first lateral direction from a midsagittal plane of the second vertebra. Method 300 is generally similar to methods 100 and/or 200, and may include one or more steps of methods 100, 200, 400, and 500, but is different in some respects. Additional details of method 300 are set forth below with respect to the elements of scoliosis treatment system 1000. The differences between method 300 and one or more of methods 100, 200, 400, and 500 will be described hereinafter.

In step 310, a spinal column having a lateral convex curve is identified. In step 320, a first anchor, a second anchor, and a tether is provided. In step 330, first anchor is secured to a first vertebra of the spinal column. In step 340, second anchor is secured to a second vertebra of the spinal column. Steps 310, 320, 330, 340, are each substantially similar to steps 110, 120, 130, and 140, respectively and as described above.

In step 350, a first tether end to the first anchor is slidably secured at a first tether connection location located in the first lateral direction from a midsagittal plane of the first vertebra. In an exemplary embodiment, first tether end 1306 is slidably secured to first anchor 1100 at first tether connection location 1106 in the first lateral direction from the midsagittal plane of the first vertebra. In a non-limiting example, slidably securing the first tether end 1306 to the first anchor 1100 comprises positioning the first tether end 1306 within a first cannulated shaft 1304 located at the first tether connection location 1106. Further, slidably securing first tether end 1306 to first anchor 1100 further comprises fixedly securing a tether travel stop 1310 to a distal portion of the first tether end 1306, with the first cannulated shaft 1304 between the tether travel stop 1310 and the second tether end 1308.

In step 360, a second tether end to the second anchor is secured at a second tether connection location located in the first lateral direction from a midsagittal plane of the second vertebra. Step 360 is substantially similar to step 160, as described above.

Additionally or optionally, method 300 comprises positioning a compression spring between the tether travel stop and the first cannulated shaft. In an exemplary embodiment, compression spring 1302 is positioned between tether travel stop 1310 and first cannulated shaft 1304. Still further, method 300 comprises steps of securing one or more additional anchors to one or more respective additional vertebrae located between the first vertebra and the second vertebra; and securing the tether to the one or more additional anchors. In an exemplary embodiment, one or more additional anchors (which may be similar to anchors 1100, 1200) is secured to one or more respective additional vertebrae located between the first vertebra and the second vertebra. Tether 1300 may be secured to the one or more additional anchors. In a non-limiting example, tether 1300 is secured to the one or more additional anchors by slidably securing tether 1300 to at least one of the one or more additional anchors. At least one of the one or more additional anchors comprises a respective cannulated shaft (which may be similar to cannulated shaft or rod 1304).

Method 300 may also comprise steps of securing a support anchor to a respective additional vertebra located between the first vertebra and the second vertebra; and fixedly connecting a support link between the one support anchor and one of the first anchor and the second anchor. In an exemplary embodiment, support anchor 1120, 1220 is secured to a respective additional vertebra located between the first vertebra and the second vertebra. Support link 1122 may be fixedly connected between one support anchor 1120, 1220 and one of the first anchor 1100 and second anchor 1200.

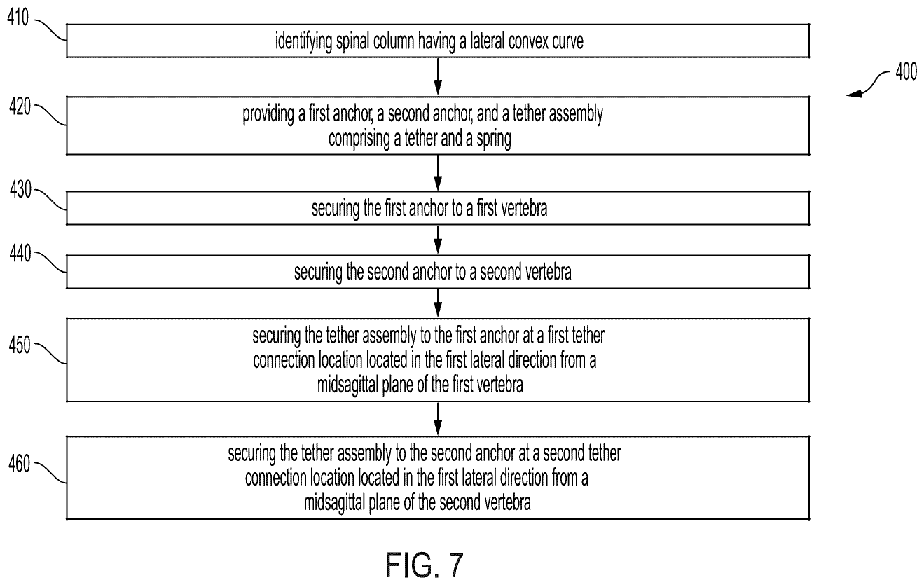

FIG. 7 illustrates another exemplary method of treating scoliosis in a patient, such as method 400, in accordance with aspects of the present invention. In particular, method 400 includes one or more steps including identifying a spinal column having a lateral convex curve; providing a first anchor, a second anchor and a tether assembly comprising a tether and a spring; securing the first anchor to a first vertebra of the spinal column; securing the second anchor to a second vertebra of the spinal column; securing a first tether end to the first anchor at a first tether connection location located in the first lateral direction from a midsagittal plane of the first vertebra; and securing a second tether end to the second anchor at a second tether connection location located in the first lateral direction from a midsagittal plane of the second vertebra. Method 400 is generally similar to one or more of methods 100, 200, 300, and 500, and method 400 may include one or more steps of one or more of methods 100, 200, 300, and 500, but is different in some respects. Additional details of method 400 are set forth below with respect to the elements of scoliosis treatment system 1000. The differences between method 400 and one or more of methods 100, 200, 300, and 500 will be described hereinafter.

In step 410, a spinal column having a lateral convex curve is identified. Step 410 is substantially similar to step 110, as described above.

In step 420, a first anchor, a second anchor, and a tether is provided. In an exemplary embodiment, a first anchor, such as first anchor 1100, a second anchor, such as second anchor 1200, and a tether assembly. In a non-limiting example, tether assembly comprises a tether 1300 and a spring 1302.

In step 430, first anchor is secured to a first vertebra of the spinal column. In step 440, second anchor is secured to a second vertebra of the spinal column. In step 450, a first tether end to the first anchor is secured at a first tether connection location located in the first lateral direction from a midsagittal plane of the first vertebra. In step 460, a second tether end to the second anchor is secured at a second tether connection location located in the first lateral direction from a midsagittal plane of the second vertebra. Steps 430, 440, 450, 460, are each substantially similar to steps 130, 140, 150, and 160, respectively and as described above.

Additionally or optionally, method 400 comprises positioning a spring between the tether travel stop and the first tether connection location. In an exemplary embodiment, spring 1302 is positioned between tether travel stop 1310 and first tether connection location 1106.

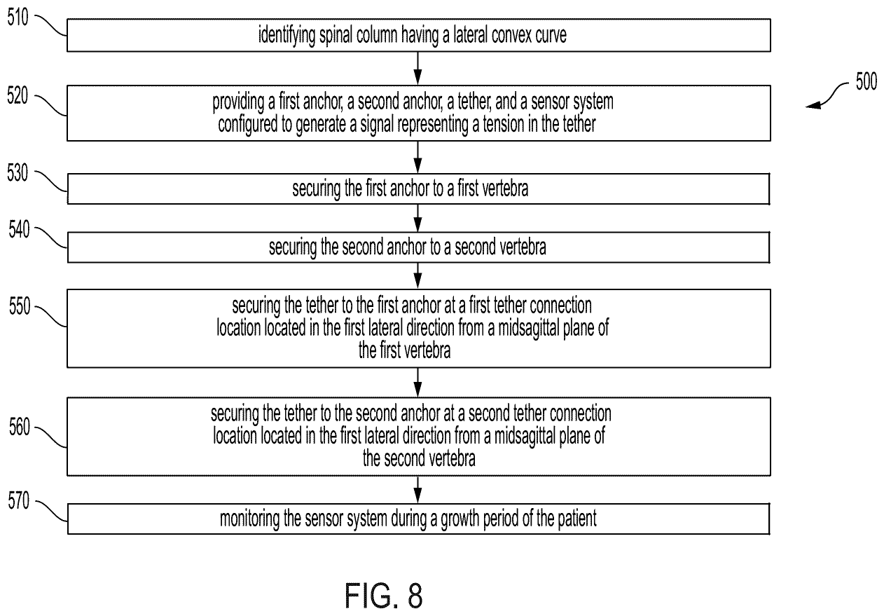

FIG. 8 illustrates another exemplary method of treating scoliosis in a patient, such as method 500, in accordance with aspects of the present invention. In particular, method 500 includes one or more steps including identifying a spinal column having a lateral convex curve; providing a first anchor, a second anchor and a tether assembly comprising a tether and a spring; securing the first anchor to a first vertebra of the spinal column; securing the second anchor to a second vertebra of the spinal column; securing a first tether end to the first anchor at a first tether connection location located in the first lateral direction from a midsagittal plane of the first vertebra; and securing a second tether end to the second anchor at a second tether connection location located in the first lateral direction from a midsagittal plane of the second vertebra. Method 500 is generally similar to one or more of methods 100, 200, 300, and 400, and method 500 may include one or more steps of one or more of methods 100, 200, 300, and 400, but is different in some respects. Additional details of method 500 are set forth below with respect to the elements of scoliosis treatment system 1000. The differences between method 500 and one or more of methods 100, 200, 300, and 400 will be described hereinafter.

In step 510, a spinal column having a lateral convex curve is identified.

In step 520, a first anchor, a second anchor, a tether, and a sensory system is provided. In an exemplary embodiment, a first anchor, such as first anchor 1100, a second anchor, such as second anchor 1200, a tether assembly, such as tether 1300, and a sensor system configured to generate a signal representing a tension in tether 1300, such as sensor system 1400.

In step 530, first anchor is secured to a first vertebra of the spinal column. In step 540, second anchor is secured to a second vertebra of the spinal column. In step 550, a first tether end to the first anchor is secured at a first tether connection location located in the first lateral direction from a midsagittal plane of the first vertebra. In step 560, a second tether end to the second anchor is secured at a second tether connection location located in the first lateral direction from a midsagittal plane of the second vertebra. Steps 530, 540, 550, 560, are each substantially similar to steps 130, 140, 150, and 160, respectively and as described above.

In step 570, the sensor system is monitored during a growth period of the patient. In an exemplary embodiment, sensory system 1400 is monitored during the growth period of the patient. In a non-limiting example, sensor system 1400 comprises one or more strain gauges 1500 attached to one or more of the first anchor 1100, the second anchor 1200, and the tether 1300. Further, sensor system 1400 comprises a wireless transmitter 1600 configured to wirelessly send the signal to a remote monitoring device 1700.

Monitoring the sensor system comprises periodically or continuously monitoring a resting tension in the tether 1300 during the growth period of the patient. Further, monitoring the resting tension in the tether 1300 comprises wirelessly communicating with a control unit (such as strain gauge 1500) operatively connected to the tether 1300. In some cases, monitoring the sensor system comprises collecting clinical data using biofeedback technology for periodic or continuous monitoring of patients, such as patients treated for instrumented spinal deformities and patients treated with one or more of the devices, methods or systems described herein. In particular, monitoring the sensor system may comprise continuously performing wireless monitoring of the implanted spinal hardware, such as anchors 1100, 1200 and/or tether 1300. The motion and/or loading throughout the day as applied by the implanted components of the scoliosis treatment system is continuously and wirelessly monitored, thereby permitting assessment of patient activity and the mechanical environment at the spinal deformity site. Non-limiting examples of clinical data to be collected may include: mean deformation amplitude per 6 hours; mean deformation rate; number of load-cycles; histogram of patient activity (load intensity distribution); or a combination thereof. The raw collected data may be processed and analyzed to assess the average influence of physiological loading variances, thereby providing a patient specific histogram (displayable via a Human Machine Interface (HMI)), for individualizing rehabilitation protocols. The collected data can be generated at high frequencies, in real-time and via remote monitoring. This allows for more reliable assessment of clinical progress of the implanted spinal hardware and supports evidence-based decision making throughout the course of the corrective intervention of the subject spinal deformity.

Additionally or optionally, method 500 comprises adjusting the tension in the tether 1300 according to a determination of the monitored resting tension.

In all of the foregoing methods, FIGS. 4, 5, 6, 7, and 8 each depicts an exemplary method comprising steps that are performed sequentially in the order recited. However, it should be understood from the description herein that one or more steps may be omitted and/or performed out of the described sequence of the process while still achieving the desired result.

### EXAMPLE

The inventors assessed the feasibility and functionality of components of the exemplary device, method of treatment of scoliosis, and scoliosis treatment systems, as well as verified any updates or improvements made. The prototype device 1000b comprised first anchor 1100, a second anchor 1200, and a tether 1300. The prototype device 1000b generally comprise components and/or operationally similar as in the embodiment described above. Tether 1300 comprised a 1.7 mm cobalt chrome flexible metal tether cable. A spring 1302 is operationally secured between tether 1300 and the first tether connection location 1106 to compress or expand as a function of tension in tether 1300.

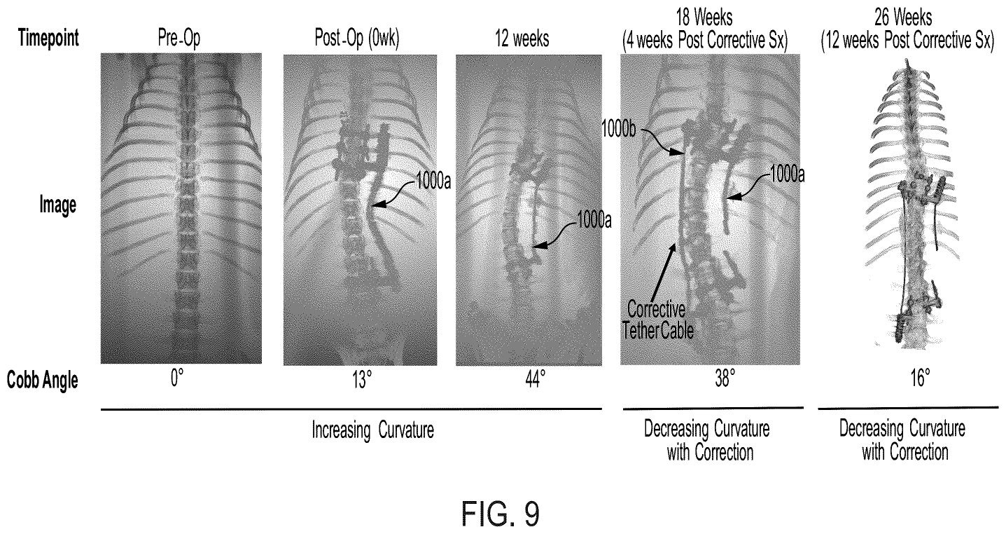

The prototype was subjected to clinical testing as detailed herein and as illustrated in FIG. 9. A prototype sample lacking spring 1302 (i.e. device 1000a) was implanted in a rapidly growing (˜10 kg) Yucatan mini-pig. In this study, tension or force of tether 1300a (as indicated by spring compression) was observed, measured, and monitored. Additionally, the stiffness or tension of the tether 1300 of device 1000a was modulated. As the mini-pig experienced a growth period, the forces of growth are restricted by force exerted by tether 1300 of system 1000a on the spine, thereby leading to development or exacerbation of scoliosis (at 44°), which can be seen at 12 weeks post-op in FIG. 9. At 14 weeks, after the initial surgery, tether 1300 of device 1000a was cut and new lateral offsets 1100, 1200 were placed along the convex side of the curve (opposite location of tether 1300 of system 1000a up to 12 weeks), with a new tether 1300 of device 1000b, which is designed in accordance with aspects of the invention to correct and reduce the curvature of the spine in view of the ongoing growth period of the patient/animal. Thus, improved or correct curvature can be observed at 4 weeks post the corrective surgery (18 weeks total), which illustrates success in achieving a decreased curvature of the spine with use of scoliosis treatment system comprising tether 1300 and spring 1302 of device 1000b. Still further, relatively more improved or correct curvature can be observed 12 weeks after the corrective surgery (26 weeks after initial surgery), such that the spinal curve had decreased to 12-16 degrees from a peak of 44 degrees (at 12 weeks).

Although the invention is illustrated and described herein with reference to specific embodiments and examples, the invention is not intended to be limited to the details shown, and all examples are non-limiting examples. Rather, various modifications may be made in the details within the scope and range of equivalents of the claims and without departing from the invention.

1. A method for treating scoliosis in a patient, the method comprising:
identifying a spinal column having a lateral convex curve extending in a first lateral direction from a midsagittal plane of the patient;
providing a first anchor, a second anchor, and a tether;
securing the first anchor to a posterior region of a first vertebra of the spinal column;
securing the second anchor to a posterior region of a second vertebra of the spinal column;
securing a first tether end of the first anchor at a first tether connection location located in the first lateral direction from a midsagittal plane of the first vertebra;
securing a second tether end to the second anchor at a second tether connection location located in the first lateral direction from a midsagittal plane of the second vertebra.

2. The method of claim 1, wherein securing the first anchor to the posterior region of the first vertebra comprises securing the first anchor to the pedicle of the first vertebra, securing the second anchor to the posterior region of the second vertebra comprises securing the second anchor to the pedicle of the second vertebra, or a combination thereof.

3. The method of claim 1, wherein the first anchor comprises a first anchor body and one or more first pedicle screws, the second anchor comprises a second anchor body and one or more second pedicle screws, or a combination thereof.

4. The method of claim 1, wherein securing the first anchor to the posterior region of the first vertebra comprises securing the first anchor to the posterior region of the first vertebra at least two locations on the first vertebra; and securing the second anchor to the posterior region of the second vertebra comprises securing the second anchor to the posterior region of the second vertebra in at least two locations on the second vertebra.

5. The method of claim 4, wherein the at least two locations on the first vertebra comprise two locations on opposite lateral sides of a midsagittal plane of the first vertebra; and the at least two locations on the second vertebra comprise two locations on opposite lateral sides of a midsagittal plane of the second vertebra.

6. The method of claim 1, wherein the first anchor comprises a first offset connector portion extending in a lateral direction from the midsagittal plane of the first vertebra to the first tether connection location; and the second anchor comprises a second offset connector extending in the lateral direction from the midsagittal plane of the second vertebra to the second tether connection location.

7. The method of claim 5, further comprising:
securing a first support anchor to a posterior region of a third vertebra of the spinal column adjacent to the first vertebra and between the first vertebra and the second vertebra;
securing the first support anchor to the first anchor to inhibit rotation of the first anchor relative to the first vertebra;
securing a second support anchor to a posterior region of a fourth vertebra of the spinal column adjacent to the second vertebra and between the first vertebra and the second vertebra; and
securing the second support anchor to the second anchor to inhibit rotation of the second anchor relative to the second vertebra.

8. The method of claim 7, further comprising:
securing the first anchor to the posterior region of the first vertebra comprises securing the first anchor to the posterior region of the first vertebra in at least two locations on the first vertebra;
securing the second anchor to the posterior region of the second vertebra comprises securing the second anchor to the posterior region of the second vertebra in at least two locations on the second vertebra;
securing the first support anchor to the posterior region of the third vertebra comprises securing the first support anchor to the posterior region of the third vertebra in at least two locations on the third vertebra; and
securing the second support anchor to the posterior region of the fourth vertebra comprises securing the second support anchor to the posterior region of the fourth vertebra in at least two locations on the fourth vertebra.

9. The method of claim 1, wherein securing the first tether end to the first anchor comprises securing a first cannulated rod to the first anchor and slidably securing the first tether end within the first cannulated rod.

10. The method of claim 9, wherein slidably securing the first tether end within the first cannulated rod comprises:
providing a spring having a first end and second end;
securing a distal portion of the first tether end to the first end of the spring;
positioning the second end of the spring between the first end of the spring and the first cannulated rod; and
positioning the first cannulated rod between the second end of the spring and the second anchor.

11. The method of claim 9, wherein securing the second tether end to the second anchor comprises securing a second cannulated rod to the second anchor and slidably securing the second tether end within the second cannulated rod.

12. The method of claim 1, further comprising generating a tensile force in the tether between the first anchor and the second anchor.

13. The method of claim 12, wherein generating the tensile force comprises installing a spring between the tether and at least one of the first anchor and the second anchor.

14. The method of claim 1, wherein the tether comprises a metal wire.

15. The method of claim 1, wherein the tether comprises a braided rope.

16. The method of claim 1, further comprising:
allowing a growth period of the patient;
adjusting a tension in the tether, adjusting a length of the tether between the first anchor and the second anchor, or a combination thereof.

17. The method of claim 1, further comprising periodically or continuously monitoring a resting tension in or a length of the tether during a growth period of the patient.

18. The method of claim 17, wherein monitoring the resting tension in or length of the tether comprises wirelessly communicating with a control unit operatively connected to the tether.

19. A method for treating scoliosis in a patient, the method comprising:
installing a tether between a first vertebra of spinal column of the patient and a second vertebra of a spinal column of the patient, wherein the tether is offset on a first lateral side of a midsagittal plane of the first vertebra and offset on a first lateral side of a midsagittal plane of the second vertebra, with the first lateral side of a midsagittal plane of the first vertebra and the first lateral side of a midsagittal plane of the second vertebra are offset in a common lateral direction relative to a midsagittal plane of the patient;
generating a variable resting tensile force in the tether by a combination of increasing a distance between the first vertebra and the second vertebra, and changing a length of a variable force member installed between the tether and the first vertebra.

20. The method of claim 19, wherein the variable force member comprises a spring mounted in compression between the tether and the first vertebra.

21. The method of claim 20, further comprising monitoring the variable resting tensile force during a growth period of the patient.

22. The method of claim 21, wherein monitoring the variable resting tensile force comprises wirelessly communicating with a control unit operatively connected to the tether.

23. The method of claim 22, wherein the control unit comprises a strain gauge connected to an anchor securing the tether to the first vertebra or the second vertebra.

24. A scoliosis treatment system comprising:
a first anchor configured to install on a posterior region of a first vertebra, the first anchor comprising:
one or more first anchor locations at which the first anchor is configured to secure to the first vertebra, and
a first offset connector portion fixedly connected to the one or more first anchor locations and extending from the one or more first anchor locations to a first tether connection location;
wherein the first anchor is configured such that, when the one or more first anchor locations are secured to the first vertebra:
the first tether connection location is positioned at a first location offset in a first lateral direction from a midsagittal plane of the first vertebra, and
the first anchor does not include any tether connection locations offset in in a second lateral direction, opposite the first lateral direction, from the midsagittal plane of the first vertebra;
a second anchor configured to install on a posterior region of a second vertebra, the second anchor comprising:
one or more second anchor locations at which the second anchor is configured to secure to the second vertebra, and
a second offset connector portion fixedly connected to the one or more second anchor locations and extending from the one or more second anchor locations to a second tether connection location,
wherein the second anchor is configured such that, when the one or more second anchor locations are secured to the second vertebra:
the second tether connection location is positioned at a second location offset in a second lateral direction from a midsagittal plane of the second vertebra, and
the second anchor does not include any tether connection locations offset in in a second lateral direction, opposite the second lateral direction, from the midsagittal plane of the second vertebra; and
a tether configured to attach to and extend between the first tether connection location and the second tether connection location.

25. The system of claim 24, wherein the one or more first anchor locations comprises at least two first anchor locations configured to secure to the first vertebra on opposite sides of the midsagittal plane of the first vertebra; and the one or more second anchor locations comprises at least two second anchor locations configured to secure to the second vertebra on opposite sides of the midsagittal plane of the second vertebra.

26. The system of claim 24, wherein first tether connection location is configured to be offset in the first lateral direction from the midsagittal plane of the first vertebra relative to all of the one or more first anchor locations when the one or more first anchor locations are secured to the first vertebra; and the second tether connection location is configured to be offset in the first lateral direction from the midsagittal plane of the second vertebra relative to all of the one or more second anchor locations when the one or more second anchor locations are secured to the second vertebra.

27. The system of claim 24, wherein the first anchor comprises a first anchor body extending from the first tether connection to at least two first anchor locations; and the second anchor comprises a second anchor body extending from the second tether connection to at least two second anchor locations.

28. The system of claim 27, wherein the at least two first anchor locations comprise respective first pedicle screw openings; and the at least two second anchor locations comprise respective second pedicle screw openings.

29. The system of claim 24, further comprising:
a first support anchor configured to install on a posterior region of a third vertebra;
a first link configured to fixedly connect between the first support anchor and the first anchor;
a second support anchor configured to install on a posterior region of a fourth vertebra; and
a second link configured to fixedly connect between the second support anchor and the second anchor.

30. The system of claim 29, wherein the first anchor comprises a first anchor body extending between at least two first anchor locations.

31. The system of claim 29, wherein the first support anchor comprises a first support anchor body extending between at least two first support anchor locations.

32. The system of claim 29, wherein the first link comprises a respective first link collar configured to secure to the first anchor body between the at least two first anchor locations and a respective second link collar configured to secure to the first support anchor body between the at least two first support anchor locations.

33. The system of claim 29, wherein the second anchor comprises a second anchor body extending between at least two second anchor locations.

34. The system of claim 29, wherein the second support anchor comprises a second support anchor body extending between at least two second support anchor locations.

35. The system of claim 29, wherein the second link comprises a respective first link collar configured to secure to the second anchor body between the at least two second anchor locations and a respective second link collar configured to secure to the second support anchor body between the at least two second support anchor locations.

36. The system of claim 24, wherein the first tether connection location comprises a first cannulated rod configured to slidable contain a first tether end.

37. The system of claim 36, further comprising a spring having a first spring end configured to fixedly secure to the tether and a second spring end configured to be positioned between the first spring end and the first cannulated rod with the first cannulated rod between the spring and the second tether connection location.

38. The system of claim 24, wherein the tether comprises a metal wire.

39. The system of claim 24, wherein the tether comprises a braided rope.

40. The system of claim 24, further comprising a sensor system configured to generate a signal representing a tension in the tether.

41. The system of claim 40, wherein the sensor system comprises one or more strain gauges or microelectromechanical systems (MEMS) sensors attached to one or more of the first anchor, the second anchor, and the tether.

42. The system of claim 40, wherein the sensor system comprises a wireless transmitter configured to wirelessly send the signal to a remote monitoring device.

43. The system of claim 24, further comprising a document comprising a set of use instructions or instructions for accessing the set of use instruction, the set of use instructions comprising instructions for:
securing the first anchor to the posterior region of the first vertebra;
securing the second anchor to the posterior region of the second vertebra; and
securing the tether to the first tether connection location and the second tether connection location.

44. A scoliosis treatment system comprising:
a first anchor configured to install on a first vertebra, the first anchor comprising:
one or more first anchor locations at which the first anchor is configured to secure to the first vertebra, and
a first offset connector portion fixedly connected to the one or more first anchor locations and extending from the one or more first anchor locations to a first tether connection location,
wherein the first anchor is configured such that, when the one or more first anchor locations are secured to the first vertebra, the first tether connection location is positioned at a first location offset in a first lateral direction from a midsagittal plane of the first vertebra;
a second anchor configured to install on a second vertebra, the second anchor comprising:
one or more second anchor locations at which the second anchor is configured to secure to the second vertebra, and
a second offset connector portion fixedly connected to the one or more second anchor locations and extending from the one or more second anchor locations to a second tether connection location,
wherein the second anchor is configured such that, when the one or more second anchor locations are secured to the second vertebra, the second tether connection location is positioned at a second location offset in a second lateral direction from a midsagittal plane of the second vertebra; and
a tether extending from a first tether end configured to be slidably secured within the first tether connection location, to a second tether end configured to be secured at the second tether connection location.

45. The system of claim 44, wherein the first tether connection location comprises a first cannulated shaft, and the tether is configured to be slidably positioned within the first cannulated shaft.

46. The system of claim 45, further comprising a tether travel stop configured to prevent a distal portion of the first tether end from passing through the first cannulated shaft towards the second tether end.

47. The system of claim 46, further comprising a compression spring located between the tether travel stop and the first cannulated shaft.

48. The system of claim 44, wherein the first anchor location comprises a threaded screw shaft and the first offset connector portion comprises a head attached to the threaded screw shaft, the head having a hole or a slot therethrough to slidably receive the first tether end.

49. The system of claim 44, wherein the first anchor comprises a first anchor body having a plurality of first connection locations configured to separately secure to the first vertebra.

50. The system of claim 44, wherein the first anchor is configured such that, when the one or more first anchor locations are secured to the first vertebra, the first anchor does not include any tether connection locations offset in in a second lateral direction, opposite the first lateral direction, from the midsagittal plane of the first vertebra.

51. The system of claim 50, wherein the second anchor is configured such that, when the one or more second anchor locations are secured to the second vertebra, the second anchor does not include any tether connection locations offset in in a second lateral direction, opposite the second lateral direction, from the midsagittal plane of the second vertebra.

52. The system of claim 44, further comprising one or more additional anchors, each additional anchor comprising:
one or more respective additional anchor locations at which the respective additional anchor is configured to secure to a respective vertebra located between the first vertebra and the second vertebra; and
a respective additional offset connector portion fixedly connected to the one or more respective additional anchor locations and extending from the one or more respective additional anchor locations to a respective additional anchor tether connection location;
wherein:
each additional anchor is configured such that, when the one or more respective additional anchor locations are secured to the respective vertebra, the respective additional anchor tether connection location is positioned at a first location offset in a first lateral direction from a midsagittal plane of the respective vertebra, and
the tether is configured to be secured at respective locations to the respective additional anchor tether connection location.

53. The system of claim 52, wherein the tether is configured to be slidably secured to one or more of the respective additional anchor tether connection locations.

54. The system of claim 53, wherein one or more of the additional anchor tether connection locations comprises a respective additional cannulated shaft, and the tether is configured to be slidably positioned within each of the additional cannulated shafts.

55. The system of claim 44, further comprising:
a support anchor configured to install on a third vertebra located between the first vertebra and the second vertebra; and
a support link configured to connect to one of the first anchor and the second anchor.

56. The system of claim 44, wherein the tether comprises a metal wire.

57. The system of claim 44, wherein the tether comprises a braided rope.

58. The system of claim 44, further comprising a sensor system configured to generate a signal representing a tension in the tether.

59. The system of claim 58, wherein the sensor system comprises one or more strain gauges or microelectromechanical systems (MEMS) sensors attached to one or more of the first anchor, the second anchor, and the tether.

60. The system of claim 59, wherein the sensor system comprises a wireless transmitter configured to wirelessly send the signal to a remote monitoring device.

61. The system of claim 44, further comprising a document comprising a set of use instructions or instructions for accessing the set of use instruction, the set of use instructions comprising instructions for:
securing the first anchor to the posterior region of the first vertebra;
securing the second anchor to the posterior region of the second vertebra; and securing the tether to the first tether connection location and the second tether connection location.

62. A method for treating scoliosis in a patient, the method comprising:
identifying a spinal column having a lateral convex curve extending in a first lateral direction from a midsagittal plane of the patient;
providing a first anchor, a second anchor and a tether;
securing the first anchor to a first vertebra of the spinal column;
securing the second anchor to a second vertebra of the spinal column;
slidably securing a first tether end to the first anchor at a first tether connection location located in the first lateral direction from a midsagittal plane of the first vertebra; and
securing a second tether end to the second anchor at a second tether connection location located in the first lateral direction from a midsagittal plane of the second vertebra.

63. The method of claim 62, wherein securing the first anchor to the first vertebra comprises securing the first anchor to a posterior region of the first vertebra and wherein securing the second anchor to the second vertebra comprises securing the second anchor to a posterior region of the second vertebra.

64. The method of claim 62, wherein slidably securing the first tether end to the first anchor comprises positioning the first tether end within a first cannulated shaft located at the first tether connection location.

65. The method of claim 64, wherein slidably securing the first tether end to the first anchor further comprises fixedly securing a tether travel stop to a distal portion of the first tether end, with the first cannulated shaft between the tether travel stop and the second tether end.

66. The method of claim 65, further comprising positioning a compression spring between the tether travel stop and the first cannulated shaft.

67. The method of claim 62, further comprising:
securing one or more additional anchors to one or more respective additional vertebrae located between the first vertebra and the second vertebra; and
securing the tether to the one or more additional anchors.

68. The method of claim 67, wherein securing the tether to the one or more additional anchors comprises slidably securing the tether to at least one of the one or more additional anchors.

69. The method of claim 68, wherein at least one of the one or more additional anchors comprises a respective cannulated shaft.

70. The method of claim 68, further comprising:
securing a support anchors to a respective additional vertebra located between the first vertebra and the second vertebra; and
fixedly connecting a support link between the one support anchor and one of the first anchor and the second anchor.

71. The method of claim 62, wherein the tether comprises a metal wire.

72. The method of claim 62, wherein the tether comprises a braided rope.

73. The method of claim 62, further comprising:
allowing a growth period of the patient; and
adjusting a tension in the tether and/or adjusting a length of the tether between the first anchor and the second anchor.

74. The method of claim 62, further comprising periodically or continuously monitoring a resting tension in or a length of the tether during a growth period of the patient.

75. The method of claim 74, wherein monitoring the resting tension in or length of the tether comprises wirelessly communicating with a control unit operatively connected to the tether.

76. A scoliosis treatment system comprising:
a first anchor configured to install on a first vertebra, the first anchor comprising:
one or more first anchor locations at which the first anchor is configured to secure to the first vertebra, and
a first offset connector portion fixedly connected to the one or more first anchor locations and extending from the one or more first anchor locations to a first tether connection location,
wherein the first anchor is configured such that, when the one or more first anchor locations are secured to the first vertebra, the first tether connection location is positioned at a first location offset in a first lateral direction from a midsagittal plane of the first vertebra;
a second anchor configured to install on a second vertebra, the second anchor comprising:
one or more second anchor locations at which the second anchor is configured to secure to the second vertebra, and
a second offset connector portion fixedly connected to the one or more second anchor locations and extending from the one or more second anchor locations to a second tether connection location,
wherein the second anchor is configured such that, when the one or more second anchor locations are secured to the second vertebra, the second tether connection location is positioned at a second location offset in a second lateral direction from a midsagittal plane of the second vertebra; and
a tether assembly extending from a first tether end configured to be secured at the first tether connection location, to a second tether end configured to be secured at the second tether connection location, wherein the tether assembly comprises:
a tether, and
a spring operationally secured between the tether and the first tether connection location to compress or expand as a function of tension in the tether.

77. The system of claim 76, wherein the first anchor location comprises a threaded screw shaft and the first offset connector portion comprises a head attached to the threaded screw shaft, the head having a hole or a slot therethrough to slidably receive the first tether end.

78. The system of claim 76, wherein the first anchor comprises a first anchor body having a plurality of first connection locations configured to separately secure to the first vertebra.

79. The system of claim 76, wherein the first tether end is slidably connected at the first tether connection location.

80. The system of claim 79, further comprising a tether travel stop fixedly secured to distal portion of the first tether end, with the first tether connection location between the tether travel stop and the second tether connection location.

81. The system of claim 80, wherein the spring is located between the tether travel stop and the first tether connection location.

82. The system of claim 81, wherein the first tether connection location comprises a first cannulated shaft, and the tether is configured to be slidably positioned within the first cannulated shaft.

83. The system of claim 76, further comprising one or more additional anchors, each additional anchor comprising:
one or more respective additional anchor locations at which the respective additional anchor is configured to secure to a respective vertebra located between the first vertebra and the second vertebra; and
a respective additional offset connector portion fixedly connected to the one or more respective additional anchor locations and extending from the one or more respective additional anchor locations to a respective additional anchor tether connection location;
wherein each additional anchor is configured such that, when the one or more respective additional anchor locations are secured to the respective vertebra, the respective additional anchor tether connection location is positioned at a first location offset in a first lateral direction from a midsagittal plane of the respective vertebra, and
wherein the tether is configured to be secured at respective locations to the respective additional anchor tether connection locations.

84. The system of claim 83, wherein the tether is configured to be slidably secured to one or more of the respective additional anchor tether connection locations.

85. The system of claim 84, wherein one or more of the additional anchor tether connection locations comprises a respective additional cannulated shaft, and the tether is configured to be slidably positioned within each of the additional cannulated shafts.

86. The system of claim 76, further comprising:
a support anchor configured to install on a third vertebra located between the first vertebra and the second vertebra; and
a support link configured to connect to one of the first anchor and the second anchor.

87. The system of claim 76, wherein the tether comprises a metal wire.

88. The system of claim 76, wherein the tether comprises a braided rope.

89. The system of claim 76, further comprising a sensor system configured to generate a signal representing a tension in the tether.

90. The system of claim 89, wherein the sensor system comprises one or more strain gauges or microelectromechanical systems (MEMS) sensors attached to one or more of the first anchor, the second anchor, and the tether.

91. The system of claim 90, wherein the sensor system comprises a wireless transmitter configured to wirelessly send the signal to a remote monitoring device.

92. The system of claim 76, further comprising a document comprising a set of use instructions or instructions for accessing the set of use instruction, the set of use instructions comprising instructions for:
securing the first anchor to the posterior region of the first vertebra;
securing the second anchor to the posterior region of the second vertebra; and
securing the tether assembly to the first tether connection location and the second tether connection location

93. A method for treating scoliosis in a patient, the method comprising:
identifying a spinal column having a lateral convex curve extending in a first lateral direction from a midsagittal plane of the patient;
providing a first anchor, a second anchor and a tether assembly comprising a tether and a spring;
securing the first anchor to a first vertebra of the spinal column;
securing the second anchor to a second vertebra of the spinal column;
securing the tether assembly to the first anchor at a first tether connection location located in the first lateral direction from a midsagittal plane of the first vertebra; and
securing the tether assembly to the second anchor at a second tether connection location located in the first lateral direction from a midsagittal plane of the second vertebra.

93. The method of claim 93, wherein securing the first anchor to the first vertebra comprises securing the first anchor to a posterior region of the first vertebra; and wherein securing the second anchor to the second vertebra comprises securing the second anchor to a posterior region of the second vertebra.

94. The method of claim 93, wherein securing the tether to the first anchor comprises slidably securing a first tether end at the first tether connection location.

95. The method of claim 95, wherein slidably securing the first tether end at the first anchor connection location comprises positioning the first tether end within a cannulated shaft located at the first tether connection location.

96. The method of claim 93, further comprising fixedly securing a tether travel stop to a distal portion of the first tether end, with the first tether connection location between the tether travel stop and the second tether connection location.

97. The method of claim 97, further comprising positioning a spring between the tether travel stop and the first tether connection location.

98. The method of claim 93, further comprising:
securing one or more additional anchors to one or more respective additional vertebrae located between the first vertebra and the second vertebra; and
securing the tether assembly to the one or more additional anchors.

99. The method of claim 99, wherein securing the tether assembly to the one or more additional anchors comprises slidably securing the tether to at least one of the one or more additional anchors.

100. The method of claim 100, wherein at least one of the one or more additional anchors comprises a respective cannulated shaft.

101. The method of claim 93, further comprising:
securing a support anchors to a respective additional vertebra located between the first vertebra and the second vertebra; and
fixedly connecting a support link between the one support anchor and one of the first anchor and the second anchor.

102. The method of claim 93, wherein the tether comprises a metal wire.

103. The method of claim 93, wherein the tether comprises a braided rope.

104. The method of claim 93, further comprising:
allowing a growth period of the patient; and
adjusting a tension in the tether assembly and/or adjusting a length of the tether assembly between the first anchor and the second anchor.

105. The method of claim 93, further comprising periodically or continuously monitoring a resting tension in or a length of the tether assembly during a growth period of the patient.

106. The method of claim 106, wherein monitoring the resting tension in or length of the tether assembly comprises wirelessly communicating with a control unit operatively connected to the tether.

107. The method of claim 106, wherein monitoring the resting tension in or length of the tether assembly comprises wirelessly communicating with a control unit operatively connected to the tether.

108. A scoliosis treatment system comprising:
a first anchor configured to install on a first vertebra, the first anchor comprising:
one or more first anchor locations at which the first anchor is configured to secure to the first vertebra, and
a first offset connector portion fixedly connected to the one or more first anchor locations and extending from the one or more first anchor locations to a first tether connection location,
wherein the first anchor is configured such that, when the one or more first anchor locations are secured to the first vertebra, the first tether connection location is positioned at a first location offset in a first lateral direction from a midsagittal plane of the first vertebra;
a second anchor configured to install on a second vertebra, the second anchor comprising:
one or more second anchor locations at which the second anchor is configured to secure to the second vertebra, and
a second offset connector portion fixedly connected to the one or more second anchor locations and extending from the one or more second anchor locations to a second tether connection location,
wherein the second anchor is configured such that, when the one or more second anchor locations are secured to the second vertebra, the second tether connection location is positioned at a second location offset in a second lateral direction from a midsagittal plane of the second vertebra;
a tether extending from a first tether end configured to be secured at the first tether connection location, to a second tether end configured to be secured at the second tether connection location; and
a sensor system configured to generate a signal representing a tension in the tether

109. The system of claim 108, wherein the sensor system comprises one or more strain gauges or microelectromechanical systems (MEMS) sensors attached to one or more of the first anchor, the second anchor, and the tether.

110. The system of claim 108, wherein the sensor system comprises a wireless transmitter configured to wirelessly send the signal to a remote monitoring device.

111. A method for treating scoliosis in a patient, the method comprising:
identifying a spinal column having a lateral convex curve extending in a first lateral direction from a midsagittal plane of the patient;
providing a first anchor, a second anchor, a tether, and a sensor system configured to generate a signal representing a tension in the tether;
securing the first anchor to a first vertebra of the spinal column;
securing the second anchor to a second vertebra of the spinal column;
securing the tether to the first anchor at a first tether connection location located in the first lateral direction from a midsagittal plane of the first vertebra;
securing the tether to the second anchor at a second tether connection location located in the first lateral direction from a midsagittal plane of the second vertebra; and
monitoring the sensor system during a growth period of the patient.

112. The method of claim 111, wherein monitoring the sensor system comprises periodically or continuously monitoring a resting tension in the tether during the growth period of the patient.

113. The method of claim 112, wherein monitoring the resting tension in the tether comprises wirelessly communicating with a control unit operatively connected to the tether.

114. The method of claim 111, further comprising adjusting the tension in the tether according to a determination of the monitored resting tension.

115. The method of claim 111, wherein monitoring the sensor system comprises periodically or continuously monitoring a spinal load throughout a loading cycle.

116. The method of claim 115, wherein the loading cycle occurs over a physical activity performed by the patient.
